(19) United States
(12) Patent Application Publication
(10) Pub. No.: US 2023/0346808 A1
(43) Pub. Date: Nov. 2, 2023

(54) CANNABINOID FORMULATIONS AND METHODS OF USE

(71) Applicant: PreveCeutical Medical Inc., Vancouver, British Columbia (CA)

(72) Inventors: Harendra PAREKH, Chapel Hill, Queensland (AU); Preeti PANDEY, Pallara, Queensland (AU)

(21) Appl. No.: 18/043,398

(22) PCT Filed: Aug. 31, 2021
(86) PCT No.: PCT/IB2021/022211

§ 371 Date: Feb. 28, 2023

(30) Foreign Application Priority Data
Aug. 31, 2020 (AU) 2020903103

(51) Int. Cl. A61K 47/34; A61K 9/00; A61K 9/06; A61K 9/107; A61K 47/26; A61K 31/00; A61K 47/10; A61K 36/185

(52) U.S. Cl. CPC A61K 31/658; A61K 9/0043; A61K 9/06; A61K 9/1075; A61K 36/185; A61K 47/10; A61K 47/26; A61K 47/34; A61K 2236/15; A61K 2236/333; A61K 2236/51; A61K 2236/53

(57) ABSTRACT

A cannabinoid-containing sol-gel formulation. In particular embodiments, the cannabinoid- containing sol-gel formulation is a thermo-responsive sol-gel formulation suitable for nasal delivery preferentially targeting the olfactory epithelium. The cannabinoid-containing sol-gel formulations comprise thermo-responsive poloxamers and/or poloxamines in addition to a plurality of micelles comprising a poloxamer and/or poloxamine, a surfactant and the cannabinoid composition. The invention also relates to a micellar formulation comprising a cannabinoid composition suitable for use in the sol- gel formulation.

### FIELD OF USE

The present invention relates to a cannabinoid-containing sol-gel formulation. In particular embodiments, the cannabinoid-containing sol-gel formulation is a thermo-responsive sol-gel formulation suitable for nasal delivery preferentially targeting the olfactory epithelium. The invention also relates to a micellar formulation comprising a cannabinoid composition suitable for use in the sol-gel formulation.

### BACKGROUND OF INVENTION

Cannabinoids such as cannabidiol (CBD) and delta-9-tetrahydrocannabinol (Δ⁹-THC) are known to have physical, psychological and emotional benefits. Cannabinoids interact with an endogenous signalling system, referred to as the endocannabinoid system (ECS), which consists of two major types of endogenous G protein-coupled cannabinoid receptors, CB1 and CB2. These receptors are located in the mammalian brain and throughout the central and peripheral nervous systems, including tissues associated with the immune system. CBD and Δ⁹-THC as well as endogenous cannabinoids, provide feedback inhibition of both excitatory and inhibitory transmission in the brain through activation of presynaptic CB1 receptors. CBD is considered non-psychotropic and has neuroprotectant properties. Manipulation of the ECS has shown promise in modulating disease processes including neurodegenerative diseases, cancer, epilepsy, emesis, pain, inflammation, multiple sclerosis, autism, obsessive compulsive disorder, amylotrophic lateral sclerosis, Parkinson’s disease, Huntington’s disease, Alzheimer’s disease, Lennox-Gastaut syndrome, Dravet syndrome, insomnia, anxiety and depression.

One way to non-invasively deliver pharmaceutical formulations directly to the brain is by the nasal route of administration where the pharmaceutical agent is deposited in the nasal cavity for example, on the olfactory epithelium. From the nasal cavity, the pharmaceutical agent can travel directly to the brain without encountering the blood brain barrier.

It would be advantageous to develop cannabis formulations that allow nose-to-brain delivery of cannabinoids for therapeutic effect in a wide variety of diseases both central and peripheral in nature.

### SUMMARY OF INVENTION

The present invention is predicated at least in part on the discovery that cannabinoid-containing compositions can be formulated as sol-gels and can be delivered to the nasal cavity, for example, to the olfactory epithelium in a manner that allows delivery of the cannabinoid composition to the brain.

In one aspect of the present invention, there is provided a thermo-responsive sol-gel composition comprising an aqueous solution comprising:
i) a thermo-responsive poloxamer and/or poloxamine, and
ii) a plurality of micelles, said micelles comprising a poloxamer and/or poloxamine, a surfactant and a cannabinoid composition, wherein the surfactant is not a poloxamer and/or poloxamine.

In another aspect of the present invention, there is provided a micellar composition comprising a cannabinoid composition, a poloxamer and/or poloxamine and a surfactant, wherein the surfactant is not a poloxamer or poloxamine.

In another aspect of the present invention there is provided a method of making a micellar composition comprising mixing an aqueous composition of poloxamer and/or poloxamine with a composition comprising at least one cannabinoid and a surfactant in a water miscible solvent, wherein the surfactant is not a poloxamer and/or poloxamine; to form a micellar composition.

In yet another aspect of the present invention, there is provided a method of making a sol-gel composition comprising:
i) mixing a first aqueous composition of poloxamer and/or poloxamine with a composition comprising at least one cannabinoid and a surfactant in a water miscible solvent, wherein the surfactant is not a poloxamer and/or poloxamine to form a micellar composition;
ii) mixing the micellar composition with a second aqueous composition of poloxamer and/or poloxamine.

In yet a further aspect of the invention, there is provided a micellar composition prepared by the method described herein.

In a further aspect of the invention, there is provided a sol-gel composition prepared by the method described herein.

### BRIEF DESCRIPTION OF FIGURES

FIG. 1 is a flow diagram for the preparation of a cannabis-infused sol-gel formulation; Part 1 is the preparation cannabinoid containing micelle powder and Part 2 is the preparation of the cannabinoid-infused sol-gel formulation.

FIG. 2 provides regression analysis of three-month stability data for cannabinoid-infused sol-gel formulations comprising CBD, CBDA and THC in formulations that are from native extracts or decarboxylated extracts and at cold temperature or room temperature.

FIG. 3 is a graphical representation of lactate dehydrogenase (LDH) release over 5 days by human ex-planted tissue treated with selected formulation in culture media (n = 3). Results are shown from a native Cannabis extract (N16), decarboxylated Cannabis extract (D16), PBS buffer (negative control) and 1% ZnSO₄ (positive control).

FIG. 4 provides photographic representations showing liquid spray droplet profile (4a) randomly dispersed throughout the nasal cavity cast and the sol-gel spray profile which preferentially coats the olfactory epithelia. The arrows indicate the region of the olfactory epithelia.

### DETAILED DESCRIPTION OF INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, the term “micelle” refers to a supramolecular assembly of molecules wherein hydrophobic portions of these molecules comprise the interior of the assembled micelle (i.e., the hydrophobic polymeric core) and hydrophilic portions of the molecules comprise the exterior of the assembled micelle (i.e., the outer hydrophilic polymeric layer). In this regard, micelles are spontaneously formed by amphiphilic compounds in water above a critical solute concentration, the critical micellar concentration (CMC), and at solution temperatures above the critical micellar temperature (CMT). There are many ways to determine CMC, including surface tension measurements, solubilization of water insoluble dye, or a fluorescent probe, conductivity measurements, light scattering, and the like.

The term “gel” as used herein refers to the physical properties of the compositions of the invention, which are generally semi-solid systems that include a liquid or liquid-like component and optionally solid particles and other components, such as carrier micelles, dispersed or disposed therein. A “gel” refers to the state of matter between liquid and solid. As such, a “gel” has some of the properties of a liquid (i.e., the shape is resilient and deformable) and some of the properties of a solid (i.e., the shape is discrete enough to maintain three dimensions on a two-dimensional surface).

The term “thermo-responsive sol-gel”, as generally used herein, refers to a composition, which undergoes a phase transition from a solution or liquid phase to a gel phase (e.g., the conversion of a liquid or flowable form with a viscosity of about 0.05 Pascal-seconds or less, to a gel or relatively semi-solid form with a viscosity of at least about 0.4 Pascal-seconds) or vice versa when the temperature is raised above or reduced below a critical value, which is referred to herein as a “gelation temperature” or “transition temperature”. Preferably the phase transition from a liquid to a gel and vice versa occurs in less than 10 minutes (e.g., 5 sec, 10 sec, 15 sec, 30 sec, 1 min, 2 min, 3 min, 4 min, 5 min, 6 min, 7 min, 8 min, 9 min, 10 min and any range therein), more particularly in less than 5 minutes and even more particularly in less than 2 minutes or less than 1 minute.

It is to be understood that, if any prior art publication is referred to herein, such reference does not constitute an admission that the publication forms a part of the common general knowledge in the art, in Australia or any other country.

### Micellar Composition

In one aspect of the present invention, there is provided a micellar composition comprising a cannabinoid composition, a poloxamer and/or poloxamine and a surfactant, wherein the surfactant is not a poloxamer and/or poloxamine.

The micellar composition is a carrier composition that allows the cannabinoid composition to be dispersed or solubilised in an aqueous solution in a sol-gel.

The cannabinoid composition may be a natural extract obtained directly by extraction of a Cannabis plant or part of a Cannabis plant, an extract that has been treated, for example by heat treatment to cause decarboxylation of at least some cannabinoids in the Cannabis extract or a synthetic composition comprising one or more isolated or synthetic cannabinoids and optionally other components.

The Cannabis plant may be any species of Cannabis plant, including Cannabis sativa Linnaeus, Cannabis indica Lam., and Cannabis ruderalis as well as hybrid combinations thereof and hemp plants. Cannabis varieties may also be bred to have varying cannabinoid profiles, for example, high CBD or high THC. As used herein, the term “Cannabis” refers to any and all of these plant varieties.

Extracts of Cannabis may be prepared by any means known in the art. The extracts may be formed from any part of the Cannabis plant, for example, from leaf, seed, trichome, flower, keif, shake, bud, stem or a combination thereof. In some embodiments, the extract is formed from the flowers and shake of a Cannabis plant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the micellar composition. In particular embodiments, any extractant may be removed by heating the extract optionally under reduced pressure (e.g. under vacuum). In some embodiments, the extract is filtered to remove particulate material, for example, by passing the extract through filter paper or a fine sieve (e.g. a sieve with pore sizes of 5 µm).

In some embodiments, the Cannabis extract is formed by applying heat and pressure to the plant material. Typically, in these embodiments, no extractant is required.

The cannabinoid fraction typically accounts for the majority of the compounds present in the Cannabis extract. In some embodiments, the Cannabis extract may comprise about 35% to about 95% by weight cannabinoids, for example, about 40% to about 90%, about 45% to about 70% or about 45% to about 55% by weight of the Cannabis extract. Other non-cannabinoid compounds that may be present include terpenes and terpenoid compounds.

Cannabinoids that have been identified in Cannabis plants include: Cannabigerol (CBG), Cannabigerolic acid, Cannabigerovarin, Cannabigerovarinic acid, (±)-Cannabichromene (CBC), (±)-Cannabichromenic acid, (±)-Cannabivarichromene, (±)-Cannabichromevarin, (±)-Cannabichromevarinic acid A, Cannabidiol (CBD), Cannabidivarin, Cannabidiorcol, Cannabidiolic acid (CBDA), Cannabidivarinic acid, Cannabinodiol, Cannabinodivarin, Δ⁹-Tetrahydrocannabinol (Δ⁹-THC/THC), Δ⁹-Tetrahydrocannabivarin (THCV), Δ⁹-Tetrahydrocannabiorcol, Δ⁹-Tetrahydrocannabinolic acid (THCA), Δ⁹-Tetrahydro-cannabivarinic acid, \A⁹-Tetrahydrocannablorcolic acid A and/or B, Cannabinol (CBN), Cannabivarin, Cannabiorcol, Cannabinolic acid A, Cannabitriol, Cannabiripsol, Cannabitetrol, Cannabielsoin, Cannabielsoic acid A, Cannabiglendol, Dehydrocannabifuran, Cannabifuran, Isotetrahydrocannabinol, Isotetrahydrocannabivarin, Cannabicyclol, Cannabicyclolic acid, Cannabicyclovarin, Cannabicitran, Cannabichromanone and Cannabicoumaronone. A comprehensive list of cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685).

In particular embodiments, the cannabinoid composition comprises at least one of CBD, THC, CBDA, THCA, THCV, CBG, CBN, CBC, or a mixture of two or more of these cannabinoids. In some embodiments, the cannabinoid composition comprises a main cannabinoid in an amount more than other cannabinoids present in the composition. For example, the main cannabinoid may be CBD or THC.

In some embodiments, the cannabinoid composition has been treated by heating to convert the CBDA and/or THCA to CBD and THC respectively by decarboxylation.

In some embodiments, the cannabinoid composition comprises CBD as the main cannabinoid. In other embodiments, the cannabinoid composition comprises THC as the main cannabinoid. In particular embodiments, CBD is the main cannabinoid.

In other embodiments, the compositions comprise a single cannabinoid compound, for example, one of CBD, THC, CBDA, THCA, THCV, CBG, CBN and CBC.

In some embodiments, the cannabinoid composition is present in the micellar composition in an amount in the range of 0.5% to 30% w/w of the micellar composition, especially about 1% to 20% w/w of the micellar composition, more especially about 1% to 10% w/w of the micellar composition.

The poloxamer may be a nonionic triblock copolymer composed of a central hydrophobic chain of polyoxypropylene (poly(propylene oxide) or PPO) flanked by two hydrophilic chains of polyoxyethylene (poly(ethylene oxide) or PEO). Such poloxamers may be linear or branched and include notably tri-blocks or tetra-blocks copolymers. Exemplary poloxamers include F87, F88, F98, F108, F38, F127 (P407), L35, P84, P85, L62, L63, L64, P65, F68, L72, P75, F77, P105, L42, L43, L44, P103, P104, P105, L81, L101, L121, L122, P123, P124, P188, P237 and P338.

It will further be appreciated that the nomenclature of poloxamers relates to their monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 407 (P407) describes a polymer containing a polyoxypropylene hydrophobe of about 4,000 g/mol with a hydrophilic polyoxyethylene block content of about 70% of the total molecular weight. Most preferred poloxamers are ones that are pharmaceutically acceptable for the intended route of administration of the gel-based composition or the sol-gel composition.

It will be appreciated that a poloxamer and/or a poloxamine that make up the micellar composition may be any physiologically acceptable poloxamer or poloxamine known in the art that is capable of micelle formation. Additionally, it is envisaged that the poloxamer and/or the poloxamine may include a plurality (e.g., 2, 3, 4, 5 etc or more) of poloxamers and poloxamines respectively.

In particular embodiments, the poloxamer is selected from the group consisting of P407, P124, P188, P237, P338 and any combination thereof. More particularly, the poloxamer suitably is or comprises P407 (also known as F127).

The term “poloxamine” denotes a polyalkoxylated symmetrical block copolymer of ethylene diamine conforming to the general type [(PEG)ₓ-(PPG)_{γ}]₂-NCH₂CH₂N-[(PPG)γ-(PEG)χ]₂. Each poloxamine name is followed by an arbitrary code number, which is related to the average numerical values of the respective monomer units denoted by X and Y. Poloxamines are typically prepared from an ethylene diamine initiator and synthesized using the same sequential order of addition of alkylene oxides as used to synthesize poloxamers. Structurally, the poloxamines generally include four alkylene oxide chains and two tertiary nitrogen atoms, at least one of which is capable of forming a quaternary salt. Poloxamines are usually also terminated by primary hydroxyl groups.

Poloxamines are commercially available in a wide range of EO/PO (ethyleneoxide (EO) / propylene oxide (PO)) ratios and molecular weights under the tradename Tetronic® (BASF). Exemplary poloxamines include T304, T701, T707, T901, T904, T908, T1107, T1301, T1304, T1307, T90R4, T150R1 and T1508, whose properties are shown in the table below.

TABLE 1

| Tetronic | Mw (Da) | EO units per block (a) | PO units per block (b) | HLB |
|---|---|---|---|---|
| 304 | 1650 | 3.7 | 4.3 | 12-18 |
| 701 | 3600 | 2.1 | 14.0 | 1-7 |
| 901 | 4700 | 2.7 | 18.2 | 1-7 |
| 904 | 6700 | 15 | 17 | 12-18 |
| 908 | 25000 | 114 | 21 | > 24 |
| 1107 | 15000 | 60 | 20 | 18-23 |
| 1301 | 6800 | 4 | 26 | 1-7 |
| 1304 | 10500 | 21.4 | 27.1 | 12-18 |
| 1307 | 18000 | 72 | 23 | > 24 |
| 90R4 | 6900 | 16 | 18 | 1-7 |
| 150R1 | 8000 | 5 | 30 | 1-7 |

As used herein, the term “block copolymer” can refer to a polymer in which adjacent polymer segments or blocks are different, i.e., each block comprises a unit derived from a different characteristic species of monomer or has a different composition of units.

In particular embodiments, the poloxamer and/or poloxamine have a molecular weight of between about 1,000 to about 20,000 (e.g., about 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000 and any range therein).

In certain embodiments, the poloxamer and/or poloxamine have a ratio EO units per block to PO units per block of between about 6:1 to about 1:6 (e.g., about 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6 and any range therein).

The poloxamer and/or poloxamine may be present in the micellar composition an amount from about 10% to about 98.5% or any range therein such as, but not limited to, about 20% to about 97%, about 30% to about 95%, or about 40% to about 90% by weight of the micellar composition. In particular embodiments, the poloxamer and/or poloxamine is present in an amount of about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 98.5% or any range therein, by weight of the carrier micelle. In particular embodiments, the poloxamer and/or poloxamine is present in an amount of about 50% to about 80% w/w of the micellar composition.

The surfactant described herein may be any as are known in the art and is suitably selected from the group consisting of a polyoxyethylated sorbitan fatty ester (i.e., a polysorbate e.g., Tween 20 (polyoxyethylene sorbitan monolaurate), Tween 40 (polyoxyethylene sorbitan monopalmitate), Tween 60 (polyoxyethylene sorbitan monostearate), Tween 80 (polyoxyethylene sorbitan monooleate), a polyoxyethylated glycol monoether (e.g., macrogol 15 hydroxystearate, polyethylene glycol (15)-hydroxystearate, polyoxyethylated 12-hydroxystearic acid (Kolliphor HS15, Solutol)), a polyoxyethylated glyceride, n-dodecyl tetra (ethylene oxide), a polyoxyethylated fatty acid, a polyoxyethylated castor oil (e.g. Cremophor EL (CrEL) or Kolliphor EL), a sucrose ester, a lauroyl macroglyceride, a polyglycolyzed glyceride and combinations thereof. In some embodiments, the surfactant comprises one or more C₁₂-C₂₆ alkene, diene or polyene, for example, a surfactant comprising a fatty acid selected from one or more of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid, especially where one or more alkenes are in the cis configuration such as myristoleic acid, palmitoleic acid, sapienic acid, oleic acid, linoleic acid, α-linolenic acid, arachidonic acid, eicosapentaenoic acid, erucic acid and docosahexaenoic acid.

In some embodiments, the surfactant is a non-ionic surfactant. In some embodiments, the surfactant is or comprises a polyoxyethylene sorbitan C₁₅-₂₁ alkene, especially a polyoxyethylene sorbitan C₁₅-C₂₁ alkene with one or more double bonds in the cis configuration. An exemplary surfactant is polyoxyethylene (20) sorbitan monooleate (e.g., Tween® 80). Preferably, the surfactant is a liquid at room temperature (e.g., at about 20° C. to about 25° C.). Without intending to be limited by theory, it is believed that the surfactant with its amphiphilic structure is responsible for further stabilising the cannbinoid-filled carrier micelles by associating with complementary components of the poloxamer and/or the poloxamine, and enhancing the retention of the therapeutic in the micelles when dispersed or disposed in the sol-gel composition or the gel-based composition, so as to improve stability and efficacy thereof.

In some embodiments, the surfactant is present in an amount of about 1% to about 50% (e.g., about 1.0%, 1.5%, 2.0%, 2.5%, 3.0%, 3.5%, 4.0%, 4.5%, 5.0%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%), or any range therein, by weight of the micellar composition, and in some embodiments from about 5% to about 40% or from about 20% to about 40% surfactant by weight of the micellar composition. In some embodiments, increasing the surfactant content can delay or increase the transition or gelation time of the sol-gel composition in which the micellar composition is incorporated, whilst decreasing the surfactant content can decrease the transition or gelation time of the sol-gel composition in which the micellar composition is incorporated. In other embodiments, decreasing the surfactant content will decrease the transition temperature of the sol-gel composition in which the micellar composition is incorporated. Alternatively, increasing the surfactant content can increase the transition temperature of the sol-gel composition of the sol-gel composition in which the micellar composition is incorporated.

In some embodiments, the surfactant has a Hydrophile-Lipophile Balance (HLB) number between about 5 and about 20 (e.g., about 5, 6, 7, 8, 9, 10, 11, 12, 13, 14, 15, 16, 17, 18, 19, 20 and any range therein). As will be understood by the skilled artisan, HLB is a numerical system used to describe the relationship between the water-soluble and oil-soluble parts of a nonionic surfactant.

The micellar composition may be made by preparing an aqueous solution of poloxamer and/or poloxamine. The cannabinoid composition may be prepared in a water miscible solvent for example, an alcoholic solution such as ethanol, to which is added the surfactant and the composition mixed to form a uniform mixture. The poloxamer and/or poloxamine is then mixed into the cannabinoid mixture with gentle stirring during which the poloxamer and surfactant self assemble into a micelle encapsulating the cannabinoid composition. The water miscible solvent may then be removed, for example, in vacuo. The resultant aqueous solution of micelles may then be frozen, for example, in liquid nitrogen, and the resulting mixture freeze-dried for a period of time suitable to provide a dry powder micellar composition comprising the cannabinoid composition. The dry powder micellar composition may be stored at -20° C. until use.

In another aspect of the present invention there is provided a method of making a micellar composition comprising mixing an aqueous composition of poloxamer and/or poloxamine with a composition comprising at least one cannabinoid and a surfactant in a water miscible solvent, wherein the surfactant is not a poloxamer and/or poloxamine; to form a micellar composition.

In some embodiments, the method further comprises removing the water and/or water miscible solvent. The water may be present from the aqueous composition of poloxamer and/or poloxamine. In some embodiments, the removal of the water and/or water miscible solvent results in the micelles being in dry powder form. The water and/or water miscible solvent may be removed by any means suitable, for example, under vacuum or by lyophilisation (freeze drying).

In particular embodiments, the micellar composition is in dry powder form.

In particular embodiments, the micelles in the micellar composition are nanomicelles. Nanomicelles have an average size, which refers to the average diameter of the micelle, that may be, for example, no greater than 1000 nanometers, no greater than 500 nanometers, no greater than 200 nanometers, no greater than 100 nanometers, no greater than 75 nanometers, no greater than 50 nanometers, no greater than 40 nanometers, no greater than 25 nanometers, or no greater than 20 nanometers. In certain embodiments, the carrier micelle of the present invention has an average size of between about 10 nm and about 500 nm, or any range therein such as, but not limited to, about 15 nm to about 400 nm, or about 30 nm to about 250 nm. In particular embodiments of the present invention, the carrier micelle has an average size of about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm or any range therein. In particular embodiments of the present invention, the micelles in the micellar composition have an average size of between about 10 nm and about 35 nm.

In other embodiments, the micelles in the micellar composition micromicelles. Micromicelles have an average size, which refers to the average diameter of the micelle, that may be, for example, greater than 1000 nanometers (e.g., 1050 nm, 1100 nm, 1150 nm, 1200 nm, 1250 nm, 1300 nm, 1350 nm, 1400 nm, 1450 nm, 1500 nm, 1600 nm, 1700 nm, 1800 nm, 1900 nm, 2000 nm and any range therein).

In particular embodiments, the micelles, whether comprising the cannabinoid composition or not, when dispersed in aqueous solution have a turbidity within the range of 1 to 10 NTU.

### Sol-Gel Formulations

In one aspect of the present invention, there is provided a thermo-responsive sol-gel composition comprising an aqueous solution comprising:
i) a thermo-responsive poloxamer and/or polxamine, and
ii) a plurality of micelles, said micelles comprising a poloxamer and/or poloxamine, a surfactant and a cannabinoid composition, wherein the surfactant is not a poloxamer and/or poloxamine.

The plurality of micelles is a micellar composition as described above. In some embodiments, the plurality of micelles or micellar composition is present in the sol-gel composition in an amount of about 6% to about 18% w/w of the sol-gel composition, especially about 9% to about 15% w/w of the sol-gel composition. In some embodiments, the cannabinoid composition is present in an amount of between about 0.1% and about 10% w/w of the total sol-gel composition, especially between about 0.5% and 5% w/w of the total sol-gel composition. In some embodiments, the surfactant in the micellar composition is present in an amount of about 2% to about 8% w/w of the sol-gel formulation, especially about 4% to about 6% w/w of the sol-gel composition.

The thermo-responsive poloxamer is any poloxamer as defined above that has thermo-responsive properties such that in an aqueous solution, the poloxomer undergoing a phase transition from a solution or liquid phase to a gel phase (e.g., the conversion of a liquid or flowable form with a viscosity of about 0.05 Pascal-seconds or less, to a gel or relatively semi-solid form with a viscosity of at least about 0.4 Pascal-seconds) or vice versa when the temperature is raised above or reduced below a critical value, which is referred to herein as a “gelation temperature” or “transition temperature”. Preferably the phase transition from a liquid to a gel and vice versa occurs in less than 10 minutes (e.g., 5 sec, 10 sec, 15 sec, 30 sec, 1 min, 2 min, 3 min, 4 min, 5 min, 6 min, 7 min, 8 min, 9 min, 10 min and any range therein), more particularly in less than 5 minutes and even more particularly in less than 2 minutes and most particularly less than 1 minute.

The thermo-responsive poloxamer and/or poloxamine may be selected from the poloxamer and poloxamines described above for use in the formation of the micellar composition. In some embodiments, the poloxamer and/or poloxamine used in the formation of the sol-gel composition is the same as the poloxamer and/or poloxamine used in the formation of the micellar composition. In other embodiments, the poloxamer and/or poloxamine used in the formation of the sol-gel composition is different from the poloxamer and/or poloxamine used in the formation of the micellar composition.

The poloxamer and/or poloxamine in the sol-gel composition may be present in an amount from about 0.5% to about 20% or any range therein such as, but not limited to, about 2% to about 15%, or about 2% to about 10% by weight of the sol-gel composition or the gel-based composition. In particular embodiments of the present invention, the thermo-responsive poloxamer is present in an amount of about 0.5%, 1%, 2%, 3%, 4%, 5%, 6%, 7%, 8%, 9%, 10%, 11%, 12%, 13%, 14%, 15% or any range therein, by weight of the sol-gel composition.

In some embodiments, the poloxamer and/or the poloxamine in the sol-gel composition is the same as the poloxamer and/or poloxamine in the micellar composition. In particular embodiments, the poloxamer in the micellar composition and in the sol-gel composition is P407. In these embodiments, the amount of P407 in the total sol-gel composition including the P407 in the micellar composition is in the range of 10% to 20% w/w of the sol-gel composition, especially about 12% to about 16% w/w of the sol-gel composition.

In some embodiments, the sol-gel composition further comprises one or more pharmaceutically acceptable components selected from a preservative (e.g., methyl paraben), a stabilising agent (polycarbophil or polyvinyl alcohol) a mechanical strength enhancer (e.g., hydroxypropyl methyl cellulose; HPMC E4M), a mucoadhesive (e.g., chitosan, polyvinyl alcohol (PVA), pentachlorophenol (PCP)) and a thickening agent/emulsifier (e.g., HPMC).Each of the aforementioned excipients (e.g., preservatives, mechanical strength enhancers, mucoadhesives, thickening agents, emulsifiers etc) may be included in the sol-gel composition or the gel-based composition at in an amount of about 0.05% to about 10% (e.g., about 0.05%, 0.1%, 0.2%, 0.3%, 0.4%, 0.5%, 1.0%, 1.5%, 2.0%, 2.5%, 3.0%, 3.5%, 4.0%, 4.5%, 5.0%, 6%, 7%, 8%, 9%, 10%), or any range therein, by weight of the sol-gel composition. Generally, such excipients can be included in the solution of poloxamer and/or poloxamine when preparing the sol-gel and/or added separately to the sol-gel composition.

In certain embodiments, the composition may include one or more pH-adjusting agents. For example, hydrochloric acid solutions or sodium hydroxide solutions may be added to adjust the pH of the composition. In certain embodiments, the pH of the composition is from about 5.0 to about 8.2 (e.g., about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2 and any range therein).

The aqueous solution may be any physiologically suitable aqueous solution including water, buffer solution, a salt solution, a saline solution, a sugar solution or a glucose solution. In particular embodiments, the aqueous solution is water.

In some embodiments, the sol-gel composition does not contain a solvent other than water. For example, in some embodiments, the sol-gel composition is substantially free or free of alcoholic solvents such as methanol, ethanol, propanol, 1,2-propanediol and the like. In some embodiments, the sol-gel composition is free of or substantially free of chromane compounds such as tocopherols. In some embodiments, the sol-gel composition is free or substantially free of cyclodextrin compounds. By “substantially free” is meant that the component is present in an amount that is less than 1%, less than 0.9%, less than 0.8%, less than 0.7%, less than 0.6%, less than 0.5%, less than 0.4%, less than 0.3%, less than 0.2%, less than 0.1%, less than 0.01%, and the like.

In particular embodiments, the thermo-responsive sol-gel composition described herein has a viscosity of less than about 0.15 Pa.s (e.g., 0.15, 0.14, 0.13, 0.12, 0.11, 0.10, 0.09, 0.08, 0.07, 0.06, 0.05 Pa,s etc and any range therein) at about 22° C. and greater than about 0.3 Pa.s (e.g., 0.3, 0.35, 0.4, 0.45, 0.5, 0.55, 0.6, 0.65, 0.7, 0.75, 0.8, 0.85, 0.9, 0.95, 1.0 Pa.s etc and any range therein) at about 30° C., 31° C., 32° C., 33° C., 34° C., 35° C., 36° C., 37° C., or any range therein. It will be appreciated that viscosity may be assessed by any means known in the art, such as with a viscometer or rheometer.

In one embodiment, the thermo-responsive sol-gel composition has a gel strength of greater than about 500 Pa at about 30° C. or at about 35° C. and more particularly greater than about 1000 Pa at about 30° C. or at about 35° C. As used herein, the term “gel strength” refers to the rheology of the gel. These viscoelastic properties of the thermo-responsive sol-gel composition can be determined using standard rheological characterization techniques that will be well known to one having ordinary skill in the art.

In some embodiments, the thermo-responsive sol-gel composition has a hardness in the range of hardness in the range of about 1 to about 25 g, especially about 3 to about 17 g, or every option in between. In particular embodiments, the dispensing device may be selected or adapted to manage the hardness of the sol-gel or the sol-gel hardness may be selected to be compatible with a specific dispensing device.

In some embodiments, the thermo-responsive sol-gel composition has an adhesiveness in the range of about 0.1 to about 1.5 mJ, especially about 0.3 to about 1.3 mJ. Higher adhesiveness values indicate better retention at mucosal surfaces.

In some embodiments, the thermo-responsive sol-gel composition has a cohesiveness in the range of about 0.3 to about 1.3 mJ, especially about 0.4 to about 1.2 mJ. Cohesiveness provides a measure of structural reformation of the sol-gels when subjected to mechanical forces or stress. A lower cohesiveness value provides better structural reformation.

The sol-gel composition is suitably capable of a sol-gel phase transition at a transition or gelation temperature of about 20° C. to about 40° C. (e.g., about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C.) and any range therein. Preferably, the transition temperature is about 22° C. to about 35° C. More preferably, the transition temperature is about 22° C. to about 28° C.

The sol-gel composition of the present invention is preferably a solution that is substantially free of particulates or suspended matter particulates at temperatures from about 2° C. to about 30° C. and more particularly about 10° C. to about 25° C. The turbidity or clarity of the compositions of the invention may be determined by any means known in the art, such as visually and turbidimetry. Suitably, the sol-gel composition has or demonstrates a level of turbidity or clarity of 50 Nephelometric Turbidity Units (NTU) (e.g., 50, 45, 40, 35, 30, 25, 20, 15, 10, 5 NTU or any range therein) or less, more particularly less than 20 NTU and even more particularly less than 10 NTU at temperatures from about 2° C. to about 30° C. and more particularly about 10° C. to about 25° C.

Suitably, the sol-gel composition is a single-phase solution, at typical storage temperatures (e.g., about 2° C. to about 20° C.), but when applied to, for example, a mucosal surface of a warm blooded subject (e.g., about 25° C. to about 37° C.) the sol-gel composition is converted to a gel that preferably possesses appropriate rheological and mechanical properties to promote retention at the site of application and ensure reproducible, sustained delivery of the therapeutic agent thereto. Possible advantages of this sol-gel composition include enhanced drug absorption and residence time at the target site, such as a mucosal surface (e.g., the nasal mucosa), and thereby allowing for reduced dosages and dosing frequencies, reduced irritation at the site of application, improved patient compliance and the avoidance of anterior leakage and post-nasal dripping of drug for nasal applications.

The gelation or transition temperature of the sol-gel composition described herein may be determined by any means known in the art, such as with a rheometer or by visual inspection. To this end, it will be appreciated that visual gelation temperatures are typically higher (e.g., about 4-5° C. higher) than equivalent or corresponding rheologically determined gelation temperatures. Accordingly, in particular embodiments the gelation temperatures recited herein are visual gelation temperatures or rheological gelation temperatures. Preferably, the gelation temperatures recited herein are visual gelation temperatures.

The sol-gel compositions of the invention may be prepared by preparing an aqueous solution of poloxamer and/or poloxamine and an aqueous composition comprising the micellar composition described above in separate compositions. The two compositions may then be mixed to prepare the thermo-responsive sol-gel composition. Alternatively, the aqueous poloxamer and/or poloxamine is mixed with a powdered micellar composition to give a homogeneous thermos-responsive sol-gel composition.

In another aspect of the invention, there is provided a method of making a sol-gel composition comprising:
i) mixing a first aqueous composition of poloxamer and/or poloxamine with a composition comprising one or more cannabinoids and a surfactant in a water miscible solvent, wherein the surfactant is not a poloxamer and/or poloxamine to form a micellar composition;
ii) mixing the micellar composition with a second aqueous composition of poloxamer and/or poloxamine.

In some embodiments, the method comprises the step of removing the water and/or water miscible solvent from the micellar composition formed at step i) to form a dry powder. The water may be present from the aqueous composition of poloxamer and/or poloxamine.

In particular embodments, the micellar composition used in step ii) is in dry powder form. The mixing at step ii) allows the powder form to be homogeneously dispersed and in some embodiments, hydrated, within the sol-gel composition.

### Delivery of Sol-Gel Composition to the Nasal Epithelium

The thermo-responsive sol-gel composition of the invention is suitable for sustained delivery of cannabinoids to the nasal cavity. In some embodiments, the nasal delivery is to the olfactory epithelium and underlying olfactory bulb and nerves or the trigeminal nerve network underlying the nasal epithelium. In particular embodiments, the thermo-responsive sol-gel composition delivers the cannabinoids to the olfactory epithelium.

In some embodiments, the thermo-responsive sol-gel composition provides sustained delivery of cannabinoids to the subject. For example, the cannabinoids may diffuse from the sol-gel composition over a period of time, for example, hours or days. The delivery device used for the delivery of the thermo-responsive sol-gel formulation may be selected to optimise the delivery of the formulation to the nasal epithelium based on the mechanical and rheological properties of the gel formulation.

Suitable nasal delivery devices include, but are not limited to, Pump 140 µL CPS (product number 31019927), Aptar Classic, Aptar Actator and Aptar gel devices from Aptar Pharma, France.

### Methods of Treatment

The thermo-responsive cannabinoid containing sol-gel compositions of the invention may be useful in treating and/or preventing diseases and disorders in which cannabinoids are known to be beneficial. For example, the thermo-responsive cannabinoid containing sol-gel compositions of the invention may be useful in treating diseases or disorders selected from neurological disorders including neurodegenerative diseases, Tourette’s syndrome, epilepsy, seizures, Lennox-Gastaut syndrome, Dravet syndrome, multiple sclerosis, Alzheimer’s disease, Parkinson’s disease, Huntington’s disease, spasticity, osteoporosis and amylotrophic lateral sclerosis; gastrointestinal disorders such as appetite loss, anorexia, cachexia, nausea, emesis, diabetes and Crohn’s disease; mood and behavioural disorders such as attention deficit disorder (ADD), attention deficit hyperactivity disorder (ADHD), stress, bipolar disorder, obsessive compulsive disorder, post-traumatic stress disorder (PTSD), anxiety and depression; pain, inflammation, arthritis, insomnia, fibromyalgia, spinal injury, phantom limb syndrome, migraine/headache, cramps, sleep apnoea, cancer, muscular dystrophy, HIV/AIDS, glaucoma, fatigue, asthma and autism.

In some embodiments, the thermo-responsive cannabinoid containing sol-gel compositions of the invention may be useful in treating or preventing viral infections such as influenza viruses, severe acute respiratory syndrome (SARS), Covid-19, Middle East Respiratory Syndrome (MERS), alpha coronavirus 229E, alpha coronavirus NL63, beta coronavirus OC43 and beta coronavirus HKU1.

In some embodiments, the sol-gel formulation delivers the cannabinoid composition to the brain, where it is active. In other embodiments, the cannabinoid composition may be distributed from the brain into the systemic circulation to have effects in peripheral diseases and conditions.

### EXAMPLES

### Example 1: Extraction of Cannabinoid Extracts

Cannabis flowers from Tower species (0.51% Tetrahydrocannabinol (THC) + tetrahydrocannabinolic acid (THCA) and 13.38% cannabidiol (CBD), cannabidioloic acid (CBDA)) and LA Confidential variety (2.89% THC + THCA and 8.83% CBD + CBDA) of Cannabis were stored at room temperature. 1-2 g was weighed and collected into a 50 mL specimen container. The sample was ground to a fine powder using a stainless steel coffee/spice grinder (Breville, NSW, Australia) at a single high speed for 3 minutes.

All processing with the extract was performed under minimal light conditions and/or using amber glassware or foil covered glassware to protect the cannabinoids in the extract. 1 g of powdered Cannabis flower was extracted by adding 10 mL of ethanol followed by sonication for 15 minutes. The sample was left suspended in the ethanolic solution for 24 hours in a closed container protected from light. The ethanolic suspension was then filtered and the solution collected and the solids discarded.

The extract solution was evaporated to dryness with a rotary evaporator (Heidolph, Scitek Australia Pty Ltd, NSW, Australia). The crude paste-like extract was stored at -20° C. When required, the extract was defrosted and the cannabinoid content by HPLC analysis by comparison with 8 commercial analytical cannabinoid and THC standards.

### Example 2: Decarboxylation of Cannabinoid Acids to Their Neutral Form and Extraction of Decarboxylated Cannabinoids

1 g of powdered Cannabis flower prepared as in Example 1, was placed on a clean watch glass and dried in a vacuum oven pre-set to 160° C. for 20 minutes, then air-cooled. The resultant ‘decarboxylated’ Cannabis flower powder was extracted in ethanol as set out in Example 1. The cannabinoid content was analysed by HPLC by comparison with 8 commercial analytical cannabinoid and THC standards.

### Example 3: Optimisation of Sol-Gel Formulations

The amount of Poloxamer P407 was determined based on the transition temperature of the sol-gel formulation. The total poloxamer P407 concentration in the overall sol-gel (nanomicelles and the sol-gel base) impacts the transition (gelation) temperature. A total concentration of 12 - 16% of final sol-gel composition with about 12% being in the nanomicelle formulation was found optimal in the sol-gel formulation. The amount of Poloxamer P407 was weighed into a 50 mL sampling container. 10 mL of cold water was added (4-8° C.) to provide a 12% w/v solution of P407 (foil covered). The mixture was stirred at 400 rpm on a magnetic stirrer in the cold room for 1 hour and then left to hydrate overnight in a refrigerator.

The amount of Cannabis extract was also investigated. Amounts corresponding to 1.0%, 1.5% and 2.0% w/w of the final sol-gel weight (typically 10 g) were investigated. The physicochemical properties of the Cannabis extract were found to impact the gelation temperature and rheomechanical properties of the sol-gel system. A ceiling concentration of Cannabis extract (whether decarboxylated or native form) was identified to be about 1% to provide a clear sol-gel formulation.

The surfactant for formation of micelles was investigated. 2% w/w, 5% w/w and 8% w/w (based on final sol-gel formulation) of polysorbate 80 (Tween® 80), polysorbate 60 and Kolliphor® HS15 were investigated.

The samples were analysed for stability upon storage. All samples generated sol-gels but some were turbid (polysorbate 60 sol-gels) and some formed high viscosity “sol” formulations which turned cloudy/opaque at room temperature or upon storage. Polysorbate 80 showed good phytocompatibility with other sol-gel excipients and was used in subsequent trials. Of the 2%, 5% and 8% polysorbate 80 sol-gel formulations, 5% polysorbate 80 provided optimal nanomicelles without any precipitation of cannabinoid extract and turbidity was less than 10 NTU.

The amount of thermoreversible polymer, poloxamer P407, for optimal physiological sol-to-gel transition temperature and appropriate rheomechanical properties such as viscosity and mucoadhesion was investigated. Concentration ranges of 0.5 to 4% w/w (for decarboxylated extract) and 0.5 to 3.5 % w/w (for native extract) were trialled. In addition, sol-gel stabilizing agents Noveon® polycarbophyil were trialled at 0.02, 0.025 and 0.1% w/w, while polyvinyl alcohol (PVA) was trialled at 0.01, 0.02, 0.04, 0.06 0.1 and 0.2% w/w. While these components were able to form sol-gels, the optimal thermoreversible polymer concentration was found to be 13-15% w/w of sol-gel composition (nanomicelle + sol-gel) complemented by 0.02% wt polycarbophil or 0.01% w/w PVA, with polysorbate 80 at 5 % w/w in the Cannabis nanomicelles. This optimal formulation was used in future experiments.

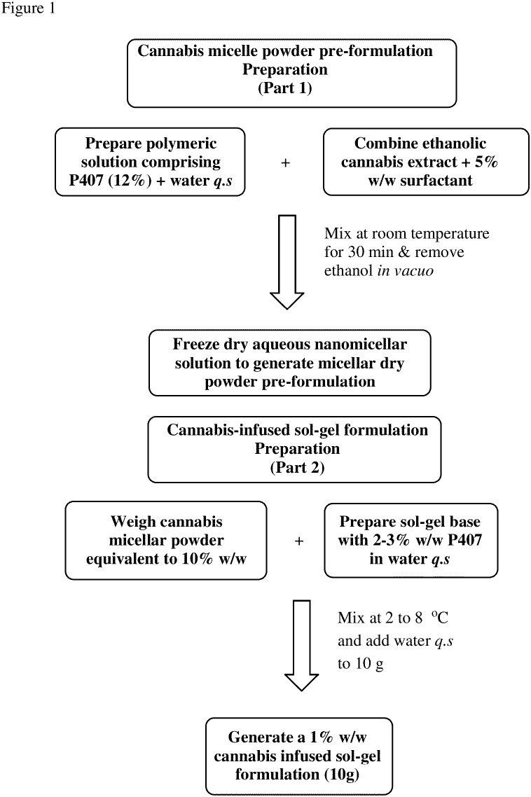

A flow diagram showing the two part formulation of nanomicelles (part 1) and nanomicelle-infused sol-gel (part 2) is shown in FIG. 1.

### Example 4: Nanomicelle Formulation With Polysorbate 80 (Tween® 80)

The required amount of Poloxamer P407 (1.2 g) was weighed into a 50 mL sampling container. 10 mL of cold water was added (4-8° C.) to provide a 12% w/v solution of P407. The mixture was stirred at 400 rpm on a magnetic stirrer in the cold room for 1 hour and then left to hydrate overnight in a refrigerator.

The paste-like Cannabis ethanolic extract prepared within the previous week, was brought to room temperature in a fumehood for 30 minutes. 100 mg of extract was weighed into an Eppendorf tube and to this was added 1-2 mL of ethanol and vortexed for 10 seconds. The solution was transferred to a 100 mL round bottom flask and 0.500 g of Tween® 80 was added and the flask shaken in a circular motion gently for 1 minute to get a uniform mixture.

The 12% w/v poloxamer solution was collected from refrigeration and transferred into the flask slowly with gentle stirring (400 rpm) for 30 minutes. The ethanol was removed in vacuo at 40° C. under 80 mbar vacuum with 100 rpm for 15-20 minutes. The resultant aqueous solution was frozen using liquid nitrogen and freeze-dried for 48 - 72 hours. The powdered extract-infused micelles were collected and stored at -20° C.

For analysis, 50 mg dry nanomicelle preparation was dispensed into a labelled centrifuge tube and mixed by vortex for 30 seconds followed by sonification at room temperature for 5 minutes to obtain a homogenised solution. The sample was centrifuged at 10000 rpm for 5 minutes. 50 µL of the sample was added to a pre-labelled centrifuge tube and 450 µL of methanol was added. The tube was vortexed for up to 5 seconds and centrifuged at 10000 rpm for 5 minutes. The sample was transferred to an amber glass HPLC vial with a polypropylene bonded cap with bonded septa and analysed by HPLC (Stationary phase: Sim-Pack XR-ODSII column (Shimadzu Scientific Instruments, NSW, Australia) with mobile phase of 0.085% phosphoric acid in water and 0.085% phosphoric acid in methanol using a gradient of 1 mL/min flow rate in a Prominence-I LC-2030 C3D Shimadzu System) together with blank (methanol), cannabinoid standards and sample, using a 10 µL sample volume.

### Example 5: Sol-Gel Preparation With Prepared Extract-Infused Nanomicelles

0.2-0.3 g (equivalent to 2-3% w/w of the final sol-gel formulation) of poloxamer P407 was added to 3 mg of preservative (typically methyl paraben, equivalent to 0.03% w/w of the final sol-gel formulation) in a required amount of cold water (up to 5 mL). The suspension was stirred for 1 hour in a cold room and then left overnight to hydrate under refrigeration.

Extract-infused nanomicelles, about 12% w/w of the final composition, were added to a separate container. The hydrated poloxamer P407 was collected from refrigeration and added to the nanomicelle powder with stirring at 500 rpm for 20 min at 4-8° C. Sufficient cold water was added to adjust the volume to prepare 10 g and a dark homogenous viscous solution was obtained.

For analysis, 50 µL sol-gel formulation was dispensed into a labelled centrifuge tube and 950 µL of methanol was added at room temperature. The samples were mixed by vortex for 5 seconds and centrifuged at 10000 rpm for 5 minutes. 100 µL of the sample was added to a pre-labelled centrifuge tube and 400 µL of methanol was added. The tube was vortexed for up to 5 seconds and centrifuged at 10000 rpm for 5 minutes. 150 µL of the sample was transferred to an amber glass HPLC vial with a polypropylene bonded cap with bonded septa and analysed by HPLC (Shimadzu Scientific Instruments, NSW, Australia) with mobile phase of 0.085% phosphoric acid in water and 0.085% phosphoric acid in methanol using a gradient of 1 mL/min flow rate in a Prominence-I LC-2030 C3D Shimadzu System) together with blank (methanol), cannabinoid standards and sample, using a 10µL sample volume.

### Example 6: Stability Analysis of Cannabinoid-Infused Sol-Gel Formulations

Samples of Cannabis-infused sol-gels containing either decarboxylated (samples D16, D17, D18) or native Cannabis extract (N14 and N16) were stored at a cold temperature (2-8° C.) or at room temperature for 3 months with analysis of content of CBD, CBDA, THC and THCA at time points 0, 1, 2 and 3 months.

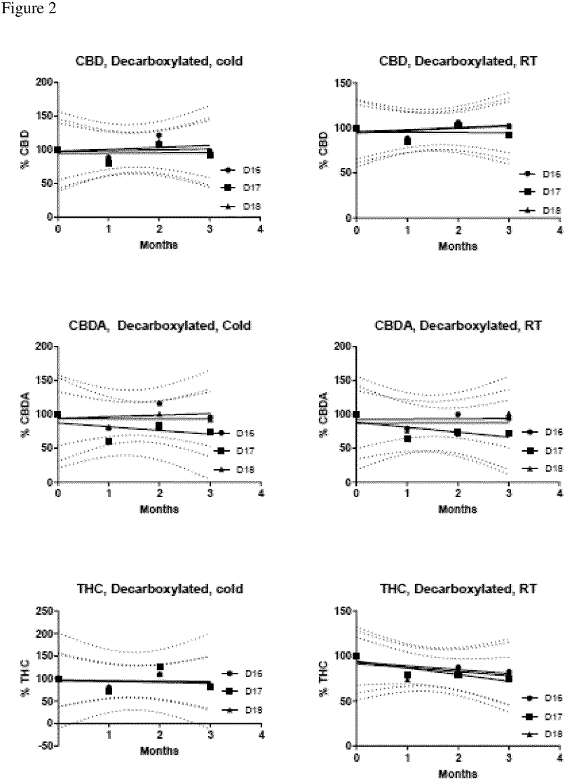

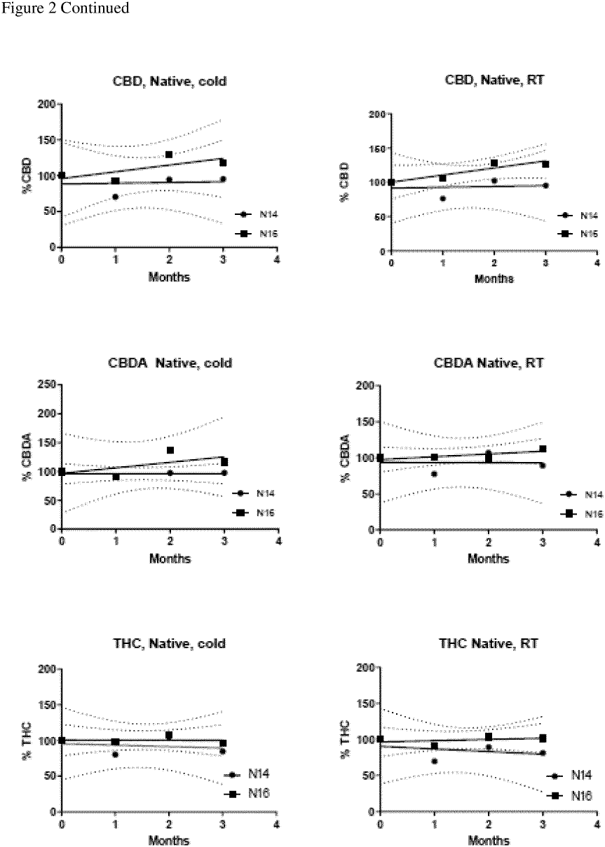

The results are shown in FIG. 2.

### Example 7: Analysis of Nanomicelle Formulations

Batches of micellar powder preformulations of native (N1 to N8) and decarboxylated (D1 to D8) were prepared together with 3 blank formulations (Cannabis free). Each formulation was prepared independently according to the process outlined in Example 4 above. Batches D1, D2 and D7 varied in cannabinoid content because of a technical error and were discarded. New batches D9 to D11 were prepared. The compositions of the nanomicelle formulations are given in Table 1:

TABLE 1

| Sample No | Micelle content | Total Batches | Batch Numbers | P407 amount | Tween® 80 amount | Extract amount |
|---|---|---|---|---|---|---|
| 1 | Blank | 3 | B1-3 | 12% w/w | 5% w/w | n/a |
| 2 | Native extract | 8 | N1-8 | 12% w/w | 5% w/w | 1% w/w |
| 3 | Decarboxylated extract | 8 | D1-8* | 12% w/w | 5% w/w | 1% w/w |
| 4 | Decarboxylated extract | 3 | D9-11 | 12% w/w | 5% w/w | 1% w/w |

*Batches D1, D2 and D7 were discarded due to a technical error

Quantitation of cannabinoids using HPLC as set out in Example 4 above, for the native samples and decarboxylated samples expressed as mg of phytocannabinoid per 2 g of dry micelle powder (equivalent to mg of cannabinoids in 100 mg of Cannabis extract) are given in Tables 2 and 3 respectively.

TABLE 2

| Sample code | CBD (mg) | CBDA (mg) | THC (mg) | THCA (mg) |
|---|---|---|---|---|
| N1 | 10.4 | 16.4 | 4.4 | 4.0 |
| N2 | 9.2 | 14.8 | 4.1 | 3.7 |
| N3 | 8.2 | 13.0 | 3.7 | 3.5 |
| N4 | 9.3 | 15.1 | 4.0 | 3.7 |
| N5 | 10.3 | 16.7 | 4.4 | 4.0 |
| N6 | 10.3 | 16.5 | 4.6 | 3.9 |
| N7 | 9.4 | 15.6 | 4.4 | 3.8 |
| N8 | 9.3 | 14.9 | 4.2 | 3.7 |

TABLE 3

| Sample code | CBD (mg) | CBDA (mg) | THC (mg) | THCA (mg) |
|---|---|---|---|---|
| D3 | 20.5 | 2.8 | 7.7 | 0.084 |
| D4 | 20.6 | 2.8 | 7.8 | 0.082 |
| D5 | 22.6 | 3.1 | 8.6 | 0.076 |
| D6 | 19.9 | 2.7 | 7.5 | 0.082 |
| D8 | 23.5 | 3.0 | 9.0 | 0.15 |
| D9 | 22.6 | 2.8 | 8.7 | 0.08 |
| D10 | 25.6 | 3.2 | ND | 0.096 |
| D11 | 21.6 | 2.7 | 8.4 | 0.076 |

ND - Not detected

The native and decarboxylated LA Confidential extract-infused micellar powders (N2, N7, N8, D3, D4 and D6) were typically light green with good colour uniformity. The micelles remained light green, with no development of wetness/melting during cold storage (2-8° C.) for 1 month.

Physicochemical properties of turbidity, size and polydispersity index were analysed and the results are given in Table 4:

TABLE 4

Physiochemical properties:

| Compound/Micelle batch | Parameters | | |
|---|---|---|---|
| | Turbidity (NTU) | Size ± SE (nm) | Polydispersity index ± SE |
| N2 | 1017 | 17.7 ± 0.15 | 0.258 ± 0.05 |
| N7 | 1.99 | 24.8 ± 4.79 | 0.363 ± 0.006 |
| N8 | 2.01 | 27.6 ± 1.77 | 0.106 ± 0.007 |
| D3 | 1.67 | 17.9 ± 1.16 | 0.234 ± 0.03 |
| D4 | 1.56 | 22.9 ±0.03 | 0.159 ± 0.05 |
| D6 | 1.33 | 18.9 ± 1.01 | 0.248 ± 0.05 |
| B1 (control) | 2.03 | 15.3 ± 0.15 | 0.197 ± 0.01 |

Assessment of turbidity showed levels less than 2 NTU which was in agreement with the blank, Cannabis free micelles indicating that the size of visually undetectable particles upon dispersion in water fell within the accepted range for colloidal particles of 1 to 10 NTU.

### Example 8: Gelation and Rheomechanical Analysis of Sol-Gel Formulations

The appearance of sol-gel formulation on storage was evaluated based on the qualitative assessment of visual inspection. Native and decarboxylated sol-gels were liquid-like fluids upon storage at 4 to 15° C., viscous liquids at temperatures between 15 and 22° C. and gels at temperatures between 22 and 35° C. The sol-gels were clear and bright with no visually detectable particulates or precipitate when products were inspected at cold temperature and room temperature using a bright-white light.

Gelation temperatures are given in Table 5:

TABLE 5

Gelation temperatures:

| Sol-Gel | Gelation Temperature (°C) |
|---|---|
| N14 | 23.1 ± 0.32 |
| N16 | 22.9 ± 0.54 |
| D16 | 24.9 ± 0.99 |
| D17 | 23.4 ± 0.89 |
| D18 | 22.4 ± 0.26 |
| Blank | 34 ± 0.3 |

Thermoresponsive properties were evaluated through oscillatory testing using temperature ramps and the energy store (storage or elastic modulus, G″ (Pa)) and the energy recovered (loss or viscous modulus, G′ (Pa)) were measured. At temperatures below 20° C., native and decarboxylated sol-gels showed low storage modulus values, which is typical behaviour of liquid-like fluids, and high loss modulus so G′ > G″. Above 20° C., storage and loss modulus values tended to increase up to the temperature at which the storage modulus was higher than the loss or viscous modulus where G″ > G′. This is indicative of gel formation. The cross-over technique was used to determine the Tsol-gel (or transition) temperature and averaged values.

Mechanical properties of the sol-gels were also examined and the results are shown in Table 6. Hardness provides information relating to integrity of the sol gel, adhesiveness is an indication of retention at mucosal surfaces and cohesiveness measures the structural reformation of a sol-gel when subjected to mechanical force or stress.

TABLE 6

Mechanical characterisation of sol-gels

| Sol-gel | Hardness (g) | Adhesiveness (mJ) | Cohesiveness (mJ) |
|---|---|---|---|
| N18 | 5.60 ± 0.27 | 0.30 ± 0.05 | 0.86 ± 0.03 |
| N19 | 4.16 ± 0.59 | 0.12 ± 0.04 | 0.72 ± 0.04 |
| N20 | 3.66 ± 0.59 | 0.19 ± 0.01 | 0.48 ± 0.17 |
| D20 | 21.6 ± 2.36 | 1.29 ± 0.24 | 0.74 ± 0.19 |
| D21 | 16.1 ± 2.36 | 0.75 ± 0.10 | 1.07 ± 0.07 |
| D22 | 11.8 ± 1.57 | 0.59 ± 0.13 | 1.06 ± 0.03 |

### Example 9: Quantitation of Cannabinoids in Sol-Gel Formulations

Native and decarboxylated cannabinoid sol-gel samples were analysed by HPLC as set out in Example 5 above for quantitation analysis. The results are shown in Table 7.

TABLE 7

Quantitation of cannabinoids in sol-gel formulations

| Sol-gel | CBD (gm/mL) | CBDA (mg/mL) | THC (mg/mL) | THCA (mg/mL) |
|---|---|---|---|---|
| N14 | 1.06 | 1.30 | 0.51 | 0.35 |
| N16 | 0.84 | 1.07 | 0.44 | 0.21 |
| D16 | 1.74 | 0.560 | 0.80 | ND |
| D17 | 1.79 | 0.700 | 0.81 | ND |
| D18 | 1.74 | 0.550 | 0.80 | ND |

ND - not detected

### Example 10: Toxicity Evaluation and Ex-Vivo Deposition of Sol Gel in Human Nasal Mucosal Tissue

The nasal cavity and its mucosal lining form an essential barrier and route for transport of pharmaceuticals directly to the brain. Development of nose-to-brain formulations require careful evaluation of metabolic and pathological changes to nasal mucosa as a means of assessing the safety of formulations. Lactate dehydrogenase (LDH) is an oxidoreductase enzyme found in living cells that is released into the cytoplasm upon cell lysis that is under stressor conditions. LDH is detected via a colourimetric cytotoxicity assay that provides a measure of membrane integrity, with high titres of LDH being an indicator of acute cellular damage/toxicity (Lim et al, International Forum of Allergy & Rhinology, 2012, 2(1): p 63-68). Histopathological examination is a complementary assessment, revealing biological characteristics of cells or non-structural tissues where morphological changes can be correlated to toxicity of formulations. Acute toxicity was assessed in freshly explanted human nasal tissue. Zinc sulfate, a well-known and reported naso-toxic agent was used as a control.

### Tissue Preparation

Freshly excised healthy human nasal mucosa was obtained from patients undergoing sinus surgery at the Greenslopes Hospital, Brisbane, Australia. Tissue specimens were collected principally from the superior nasal septum and used immediately. Human specimens were dissected, connective tissue, debris or blood were removed utilising a 15 blade scalpel and divided into 2-3 mm x 2-3 mm x 1-2 mm sections. The tissue was immediately placed in a 24-well culture system with the mucosal side exposed and washed 3 times with 300 µL of culture media comprised of 50% (high glucose)

Dulbecco’s Modified Eagle Medium (DMEM), 25% Hank’s solution, 50 UmL penicillin G and 40 µg/mL streptomycin (Invitrogen, Carlsbad, CA). Culture media (300 µL) was placed in each well and the plates incubated at 37° C. with 5% CO₂. Culture media was replaced every 24 hours for 5 days leading to stabilisation of LDH levels prior to tissue treatment.

### Tissue Treatment

After stabilisation of LDH levels, the tissue was treated with 2 µL of freshly prepared (within 2 days) sol-gel formulations, a native Cannabis infused formulation (N16) and a decarboxylated Cannabis infused formulation (D16), 1% ZnSO₄ (positive control) or PBS pH 7 (negative control) in triplicate. The samples were incubated at 37° C. with 5% CO₂. The media was harvested and replaced daily for five days. The harvested media was stored at -37° C. prior to analysis at the end of the 5 day study.

LDH was used as the biomarker and analysed using the CytoTox 96® Non-Radioactive cytotoxicity assay kit following manufacturer instructions (Promega, Madison WI). Each day of the study, a sample of culture medium (50 µL) was removed from each well and mixed with 50 µL of kit substrate. The samples were incubated at room temperature for 30 minutes and the reaction stopped by adding 50 µL of stop solution. After the end of the study, the absorbance of each sample was obtained at 490 nm using FLUOstar Omega Spectrophotometer (BMG Labtech, Offenburg, Germany). The extracellular LDH level was calculated as changes in the LDH levels of tissues treated with a formulation versus tissues treated with PBS. Statistical differences were analysed by one-way ANOVA followed by Dunnet’s multiple comparison test using GraphPad Prism software (v 7.03).

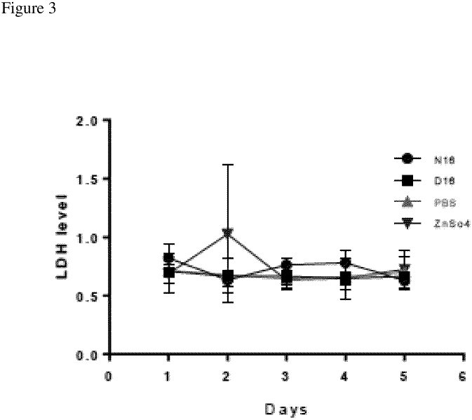

The results are shown in FIG. 3. The extracellular LDH levels form the formulations do not appear to be materially different from the negative control (PBS) at any time point (p > 0.05). The positive control caused a significant elevation in LDH levels at day 2 indicated cellular damage upon exposure to ZnSO₄.

### Tissue Staining

Untreated tissue samples and post-treated tissue samples from the end of the study were preserved from each well for histological evaluation. Each sample was fixed using 4% formaldehyde and stored at 4° C. overnight. On the following day, tissues were dipped in 20% sucrose overnight and embedded in OCT, frozen at -20° C. and cryo-sectioned into slices. Staining was performed with hematoxylin and eosin for 1 minute and stained samples dehydrated in ethanol, defatted in xylene and cover-slipped for histological examination.

Tissues were examined for morphological changes. Epithelial and sub epithelial layers of the nasal mucosa appear to be intact and unaffected in the Cannabis extract sol-gel and PBS buffer applied samples. In contrast, nasal tissue treated with 1% w/v ZnSO₄ (positive control) displayed gross cell and tissue structural damage consistent with elevated LDH levels detected in the aforementioned assay.

### Example 11: Ex Vivo Deposition of Cannabis Infused Sol-Gels

Cannabis infused-sol-gels were designed with the delivery of cannabinoids via the nasal cavity/nose-to-brain route in mind. That said key factors that impact efficacy via this route include the delivery to the olfactory epithelium as opposed to the respiratory (cavity) region, longer retention time at the nasal mucosal surface, penetration enhancement of the actives through the nasal epithelia, and a reduction in drug metabolism in the nasal cavity. The use of freshly excised human tissue to study absorption, deposition and metabolism has shown to be well-suited for these studies [Koch and Merkle, In vitro Test Systems for Drug Absorption and Delivery, C-M. Lehr, Editor, 2002, Thailor and Francis, London UK, p228-252]. The experimental setup to quantify drug deposition to explanted nasal mucosa tissue as a reliable preliminary indicator of formulation performance and their potential to access the CNS via the olfactory route, thus bypassing the blood-brain barrier is described below.

Ex vivo permeation studies were carried out using vertical Franz-diffusion apparatus equipped with temperature-controlled jackets set at 34° C. ± 1° C. Nasal mucosa was taken from middle-turbinate of patients undertaking surgery at Greenslopes Private Hospital. Mucosal tissue was cleaned of blood and bone, then immersed in phosphate-buffered saline (PBS; pH 7.4) at room temperature. The prepared nasal membrane was then sandwiched between donor and receptor cells with the dorsal side of the tissue facing the donor compartment and the ventral side facing the receiver compartment. The receptor cells were filled with 12 mL of PBS and then 300 µL of native or decarboxylated sol-gel samples placed in the donor compartment, ensuring that formulations spread over the entire tissue. To prevent evaporation of the samples, donor cells were covered with Parafilm®. Samples from the receptor cell were taken and volume replaced with PBS at 0, 1, 2, 4, 6 and 8-hours following sol-gel application. Collected tissue samples were then rinsed with MilliQ water after 8 hours, blotted dry with filter paper, wrapped with aluminium foil to protect from light and stored at -80° C. until required for further analysis.

On the day of analysis, the tissue samples were allowed to thaw and were blotted dry using filter paper, cut into small pieces, frozen with liquid nitrogen and ground with a mortar and pestle. Cannabis was extracted from the powdered tissue with ethanol (1 mL) and gently agitated for 12 hours at room temperature. Samples were then transferred to Eppendorf tubes and centrifuged at 10000 rpm for 10 minutes. The supernatant was collected and 100 µL of acetonitrile added to each tube then supernatant collected and again 100 µL was added and stored at -20° C. overnight. The following day each sample was allowed to thaw and centrifuged at 10000 rpm for 10 minutes with supernatant collected and prepared for quantitative analysis using the HPLC method described above in Example 5. Samples of Cannabis sol-gels from native (N22 and N23) and decarboxylated (D19 and D23) extracts were used in this study.

The tissue deposition of cannabinoids was measured per gram of nasal mucosa, with studies based on tissue availability from patients undergoing sinus surgery, and key cannabinoids detected presented in Table 8. The data shown is an average of the 2 native or 2 decarboxylated batches. The amount of sol-gel administered (300 µL) accounts for 3000 µg of cannabinoid content which is a surplus amount (based on micelles, this approximately correlates to the following: for native sol-gels maximum of 4950 µg CBDA, 1200 µg THCA; for decarboxylated sol-gels maximum of 960 µg CBDA, 45 µg THCA).

TABLE 8

Deposition of key cannabinoid-infused sol-gel formulations into explanted nasal mucosal tissue

| Compound | Deposition of key cannabinoids in µg/g of nasal tissue | |
|---|---|---|
| | Decarboxylated (average of batches D19 & D23) | Native (average of batches N22 & 23) |
| CBD | 3.56 ± 1.67 | 3.47 ± 1.53 |
| CBDA | 10.9 ±1.81 | 3.14 ± 2.98 |
| THC | 4.19 ± 2.20 | 3.54 ± 1.74 |
| THCA | 11.3 ± 10.2 | 5.73 ± 0.51 |

In relation to the interpretation of the deposition data the following is noted. The native (N22 and N23) and decarboxylated (D19 and D23) batches were not assessed for cannabinoid quantitation upon preparation so no direct comparisons can be made between the initial cannabinoid amounts in the sol-gel formulations and the deposited cannabinoids amounts in the mucosal tissue. In addition, the deposited cannabinoid amounts may not be completely predicted based on the amount of cannabinoids present in the formulation. There are several factors that may influence the cannabinoid deposition including: the amount of cannabinoids infusing through the tissue; their lipophilic nature and chemistry; pH; binding to proteins in tissues; their diffusion and flux rates; and the amount of cannabinoids diffusing from donor to receptor compartments across the mucosal tissue.

Eight hours post-application, the levels of CBD and THC quantified in the tissue were in close agreement for decarboxylated (3.56 and 4.19 µg/g) and native (3.47 and 3.54 µg/g) sol-gels. In contrast, CBDA and THCA deposition from decarboxylated samples were found to be much higher, at 10.9 and 11.3 µg/g of tissue, respectively. In contrast, the native sol-gels delivered only a fraction of CBDA and THCA (3.14 and 5.73 µg/g of tissue) of the decarboxylated sol-gels. However, it should be noted that in the case of THCA only, there was high variability (SEM ± 10.2 µg/g of tissue; Table 8) in the levels detected, which requires further investigation. These results suggest that the complex chemical nature and also thermal treatment of extract (native-to-decarboxylate conversion) infused into sol-gels influences tissue deposition behaviour of cannabinoids acids in-particular.

The key differences may stem from a substantial loss of volatile oils, especially terpenes (which are known as permeation enhancers) and other similar components lost from the decarboxylated extract during heat treatment, which wouldn’t be the case with native extract sol-gels. The influence of the many additional components in the native extract may for example delay the diffusion process of the acid cannabinoid forms into the nasal explant tissue and/or these components may behave as permeation enhancers aiding the diffusion of other components. The loss of terpenes may facilitate more THCA deposition and protein binding or in other words terpenes present in native extract with greater relative concentration of CBDA/THCA increases permeation/diffusion rates, leading to a greater amount being detected in the receptor compartment. Furthermore, acid forms may be more amenable to protein binding compared with neutral forms in the tissues, which would reduce their bioavailability, while neutral forms are more lipophilic with faster membrane diffusion rates.

Overall, the native formulations show consistent deposition of all key cannabinoids (Table 8), while this is clearly disrupted in the ‘heat-treated’ decarboxylated extract infused sol-gels, with higher levels (circa. 3-fold; Table 8) of CBDA detected. These tissue deposition studies highlight sol-gels as a nasal drug delivery system for Cannabis extracts, having shown an ability to deposit key cannabinoids in a quantifiable manner into explant nasal mucosa.

### Example 12: Nasal Spray Device Profiling Targeting Olfactory Epithelium

The “Pump 140 µL CPS” device (product number 31019927) was trialled, using a blank sol-gel formulation possessing the same rheological characteristics (i.e. gelation temperature, viscosity) as the Cannabis-micelle infused sol-gels. The trial sol-gel formulation was coloured green with an inert food dye to enhance visualisation.

### Experimental Set-Up

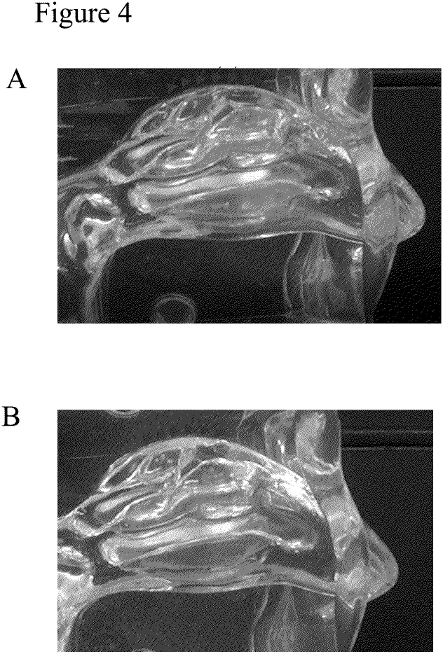

A transparent nasal cavity cast (model LM005, Koken, Japan) was mounted to an in-house fabricated suction cup with spout modified to precisely cover and seal the posterior opening of the nasal cast. The spout was then connected via plastic tubing to a diaphragm pump and the cast was wall-mounted between 2 brackets to ensure the 2 left and right hemispheres formed a tight seal. The entire set-up was established in a temperature-controlled room, pre-set to circa. 34° C., the typical nasal cavity temperature. Using the diaphragm pump air was pulled into the cavity through both nostrils at a steady rate, which was used to mimic nasal inhalation. The sol-gel was loaded into the device and sprayed into one nostril (140 µL) with simulated nasal inhalation, with the spray plume generated recorded. The still images in FIG. 4 show the resultant deposition profiles achieved with one actuation of the device using an aqueous spray (FIG. 4, left image) and separately, the sol-gel formulation (FIG. 4, right image).

### Results

As depicted in FIG. 4, traditional aqueous sprays deliver solutions randomly throughout the nasal cavity cast, with some localised pooling of the liquid, which is accompanied by notable anterior and posterior leakage. In contrast the sol-gel formulation, when delivered via the same CPS device rapidly transformed from a liquid to a gel within the cast and the inspired air deposited the sol-gel to the roof of the nasal cavity, where it effectively and comprehensively coated the target olfactory epithelium. This demonstration clearly highlights the CPS device as being suitable for the olfactory-targeted delivery of ‘viscous’ sol-gels.

1. A thermo-responsive sol-gel composition comprising an aqueous solution comprising:
i) a thermo-responsive poloxamer and/or poloxamine, and
ii) a plurality of micelles, said micelles comprising a poloxamer and/or poloxamine, a surfactant and a cannabinoid composition, wherein the surfactant is not a poloxamer and/or poloxamine.

2. The thermo-responsive sol-gel composition according to claim 1 wherein the cannabinoid composition is a Cannabis extract.

3. The thermo-responsive sol-gel composition according to claim 1, wherein the cannabinoid composition comprises one or more cannabinoid compounds.

4. The thermo-responsive sol-gel composition according to claim 1, wherein the cannabinoid composition is a cannabidiol (CBD) rich cannabinoid composition.

5. The thermo-responsive sol-gel composition according to claim 1, wherein the cannabinoid composition is present in an amount in the range of about 0.1 % w/w to about 10% w/w of the total sol-gel composition.

6. The thermo-responsive sol-gel composition according to claim 1 having a viscosity of:
i) less than 0.15 Pa.s at about 22° C.; and
ii) greater than about 0.3 Pa.s at about 30° C. or about 35° C.

7. The thermo-responsive sol-gel composition according to claim 1, wherein the sol-gel formulation has a gel strength of greater than about 500 Pa at about 30° C. or about 35° C.

8. The thermo-responsive sol-gel composition according to claim 1, wherein the sol-gel formulation has a hardness in the range of about 1 to about 25 g.

9. The thermo-responsive sol-gel composition according to claim 1, wherein the sol-gel formulation has an adhesiveness in the range of about 0.1 to about 1.5 mJ.

10. The thermo-responsive sol-gel composition according to claim 1, wherein the sol-gel formulation has a cohesiveness in the range of about 0.3 to about 1.3 mJ.

11. The thermo-responsive sol-gel composition according to claim 1, wherein the thermo-responsive poloxamer is selected from the group consisting of P407, P124, P188, P237, P338 and a combination thereof.

12. (canceled)

13. The thermo-responsive sol-gel composition according to claim 1, wherein the surfactant is selected from the group consisting of polyoxyethylated sorbitan fatty ester, a polyoxyethylated glycol monoether, a polyoxyethylated glyceride, n-dodecyltetra (ethylene oxide), a polyoxyethylated fatty acid, a polyoxyethylated castor oil, a sucrose ester, a lauroyl macroglyceride, a polyglycolyzed glyceride and any combination thereof.

14. The thermo-responsive sol-gel composition according to claim 1, wherein the surfactant comprises a C12-C26 alkene, diene or polyene.

15. The thermo-responsive sol-gel composition according to claim 1, wherein the surfactant is polyoxyethylene sorbitan monooleate.

16. A micellar composition comprising a cannabinoid composition, a poloxamer and/or poloxamine and a surfactant, wherein the surfactant is not a poloxamer and/or poloxamine.

17. A method of making the micellar composition according to claim 16, said method comprising mixing an aqueous composition of poloxamer and/or poloxamine with a composition comprising at least one cannabinoid and a surfactant in a water miscible solvent, wherein the surfactant is not a poloxamer and/or poloxamine; to form a micellar composition.

18. The method of claim 17 further comprising the step of removing water and/or water miscible solvent from the micellar composition to form a powdered micellar composition.

19. A method of making a sol-gel composition comprising:
i) mixing a first aqueous composition of poloxamer and/or poloxamine with a composition comprising at least one cannabinoid and a surfactant in a water miscible solvent, wherein the surfactant is not a poloxamer and/or poloxamine, to form a micellar composition;
ii) mixing the micellar composition with a second aqueous composition of poloxamer and/or poloxamine.

20. The method according to claim 19 further comprising the step of removing the water and/or water miscible solvent from the micellar composition formed at step i) to form a dry powder.

21. The method according to claim 19 wherein the micellar composition used in step ii) is a dry powder composition.
